(19) United States
(12) Patent Application Publication
(10) Pub. No.: US 2024/0343888 A1
(43) Pub. Date: Oct. 17, 2024

(54) ADDITIVE AND A PROCESS FOR ITS PREPARATION THEREOF

(71) Applicant: ATC TIRES PVT. LTD., Vikhroli (W), Mumbai (IN)

(72) Inventors: C. HARIMOHAN, Tirunelveli (IN); Partheban MANOHARAN, Tirunelveli (IN)

(21) Appl. No.: 18/682,297

(22) PCT Filed: Nov. 2, 2021
(86) PCT No.: PCT/IN2021/051046

§ 371 Date: Feb. 8, 2024

(30) Foreign Application Priority Data
Aug. 13, 2021 (IN) 202121036690

(51) Int. Cl. C08K 5/17; C07C 213/06; C07C 219/06; C08K 3/22; C08K 3/30; C08K 3/34; C08K 3/36; C08K 5/09; C08K 5/18; C08K 5/31; C08K 5/47; C08K 13/02; C08L 7/00; C08L 9/06

(52) U.S. Cl. CPC C08K 5/175; C07C 213/06; C07C 219/06; C08K 3/34; C08K 3/36; C08K 13/02; C08L 7/00; C08L 9/06; C08K 2003/2296; C08K 3/30; C08K 5/09; C08K 5/18; C08K 5/31; C08K 5/47

(57) ABSTRACT

The present invention relates to an additive comprising stearic acid and ethanolamine. The invention also relates to a process for the preparation of said additive. The present additive significantly improves the silica filler dispersion of rubber compositions and is able to produce said compositions with lower dosage zinc oxide.

### FIELD OF INVENTION

The present invention relates to an additive comprising stearic acid and ethanolamine. The invention also relates to a process for the preparation of said additive. The present additive significantly improves the silica filler dispersion of rubber compositions and is able to produce said compositions with lower amount of zinc oxide.

### BACKGROUND OF INVENTION

The most desirable characteristic for tires is to have good traction both on wet and dry surfaces. Traditionally, the same was achieved by including carbon black and inorganic filler (Silica) in tire compositions which further reduce rolling resistance. The rolling resistance of the tire affects vehicle fuel consumption, so a low rolling resistance tire result in a saving on the overall running cost of the vehicle.

Inorganic filler (Silica) is conventionally used to produce mechanical goods and now is increasingly used in tire tread compounds. However, the large number of hydroxyl groups on the silica surface leads to strong filler-filler interactions and reduced interaction with non-polar rubbers. In order to avoid the same, silane coupling agents or treating the silica surface or introducing hydroxyl group in polymer chain have been practically used in silica-filled compounds to improve silica dispersion and interactions between silica and rubber. Further, the mixing conditions have been optimized in order to achieve a good level of silanization and other properties.

In addition to above, during vulcanization of the rubber products, zinc oxide (ZnO) is considered to be the most efficient activator which is currently employed in the worldwide rubber production. However, zinc consumption is a worldwide environmental concern, and the automotive industry, as a main contributor, is under increasing pressure to decrease its share.

In this regard, reference may be made to U.S. Pat. No. 9,598,563B2 which relates to improving the silica dispersibility in a rubber composition. Said Patent proposed to use a silica which has been surface-treated with a silane coupling agent. In addition to that polymer having (meth) acrylate hydroxyl group have been added in order to improve the silica dispersion.

Reference may also be made to Patent publication no. US 2018/0327573 A1 which relates to a precipitated silica reinforced rubber composition containing triethanolamine. The Patent further relates to use of triethanolamine in combination with reinforcing filler (silica) in tire tread performance.

Reference is further made to non-patent literature titled “Functionalized SBRs in Silica-reinforced Tire Tread Compounds: Interactions with filler and Zinc Oxide” by Maghami et al which mentions that the dispersion of silica in rubber compound can be interfered by the addition of ZnO due to the alkali-acidic reaction. Further, it can adversely affect the cure properties of silica reinforced compounds. Since zinc oxide is intruding into the silanization reaction, hence it became more critical when functional polymer is present in the compound. Additionally, ZnO added at a later stage of mixing has significant influence on visco-elastic properties of rubber compounds.

Therefore, uniform dispersion of silica in rubber composites is a challenge to the scientific community. The sustainability of the global economy depends on the affordability of sustainable material which necessitates economically competitive sustainable product manufacturing.

Accordingly, the applicant carried out research for finding alternative means by which silica filler dispersion can be improved and the zinc oxide amounts can be decreased in the rubber compositions.

In view of above, there exist a need in the state of the art to provide an additive, which can improve the silica filler dispersion and reduce the amount of zinc oxide. Improving silica dispersion lead to the use of more silica compounds for tires and consequently, more efficient use of energy. Further, the novel and inventive additive is able to produce exceptional rubber compositions even with lower amount of zinc oxide. The additive loaded rubber compositions have excellent physico-mechanical properties, abrasion resistance and cut and chip properties.

The increasing use of silica and additive will drive the rubber industry towards sustainable technology. Additionally, the drawbacks associated with heavy metal zinc can be curtailed.

### OBJECTS OF THE INVENTION

It is an object of the present invention to provide an additive comprising: stearic acid of formula

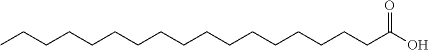 [SMILES: CCCCCCCCCCCCCCCCCC(=O)O]

and ethanolamine, selected from the group consisting of monoethanolamine, diethanolamine and triethanolamine.

A further object of the invention is to provide an additive, which improves the silica filler dispersion in rubber compositions.

Another object of the invention is to provide a process for preparation of said additive.

One more object of the present invention is provide a rubber composition having excellent properties even with lower amount of zinc oxide.

### SUMMARY OF THE INVENTION

The present invention discloses an additive comprising stearic acid and ethanolamine. The present invention also discloses an eco-friendly and simple process for preparation of said additive. The additive of present invention improves the silica filler dispersion and is able to produce rubber compositions even with lower amount of zinc oxide. The rubber compositions including the additive of present invention have excellent physico-mechanical properties, abrasion resistance and cut and chip properties.

### DESCRIPTION OF ACCOMPANYING SCHEME AND FIGURES

The accompanying drawings constitute a part of the description and are used to provide further understanding of the present invention. Such accompanying drawings illustrate the embodiments of the present invention, which are used to describe the principles of the present invention together with the description.

Scheme 1 illustrate the procedure for preparation of additive.

FIG. 1 illustrate FTIR spectrum of additive along with its components stearic acid and triethanolamine.

FIG. 2 illustrate the structure of one of the embodiment of additive.

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

The terminology used herein is for the purpose of describing particular various embodiments only and is not intended to be limiting of various embodiments. As used herein, the singular forms “a,” “an” and “the” are intended to include the plural forms as well, unless the context clearly indicates otherwise.

In one of the embodiment, the present invention provides an additive comprising: stearic acid of formula

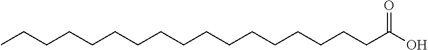 [SMILES: CCCCCCCCCCCCCCCCCC(=O)O]

and ethanolamine, selected from the group consisting of monoethanolamine, diethanolamine and triethanolamine.

In a preferred embodiment, the ethanolamine is diethanolamine

In another embodiment, the present invention provides a process for the preparation of an additive, wherein said process comprising the steps of:
a) reacting stearic acid with ethanolamine at a temperature in the range of 100-180° C. for a time period in the range of 2-5 minute to form an additive;
b) quenching of an additive of step (a) at room temperature to form an additive in solid form.

In a preferred embodiment, the temperature in step (a) for the preparation of an additive is 150° C.

In another preferred embodiment, the time period in step (a) for the preparation of additive is 3 minute.

In another preferred embodiment, the amount of stearic acid is in the range of 10% to 100% and ethanolamine is in the range of 10% to 100%. Accordingly, the stearic acid may be present from 10% to 100%. Similarly, the ethanolamine may be present from 10% to 100%.

In yet another embodiment, the present invention provides a rubber composition, comprising:
a) Additive in the range of 0.1-10 parts by weight;
b) polymers in the range of 30-100 part by weight;
c) Silica in the range of 30-90 parts by weight;
d) Silane in the range of 1-10 part by weight.

In yet another embodiment, the rubber composition wherein the polymers is selected from natural rubber, synthetic rubber and other rubbers or a combination of those rubbers.

In yet another embodiment, the rubber composition further comprises:
a) Zinc oxide in the range of 0-5 part by weight;
b) Stearic acid in the range of 0-5 part by weight;
c) TMQ in the range of 0.5-3 part by weight;
d) 6PPD in the range of 0.5-3 part by weight;
e) Sulphur in the range of 0.2-3 part by weight;
f) TBBS in the range of 0.2-3 part by weight;
g) DPG in the range of 0.2-3 part by weight; and
h) PVI in the range of 0.2-3 part by weight.

In said embodiment, the rubber composition of the present invention exhibits excellent silica dispersions and good physicochemical properties at low level of activator zinc oxide.

In another embodiment, the additive of the present invention, as and when used for reducing the amount of zinc oxide in rubber compositions to 0-5 part by weight.

The present invention is illustrated hereunder in greater detail in relation to non-limiting exemplary embodiments as per the following examples:

### EXAMPLES

The following examples are put forth so as to provide those of ordinary skill in the art with a complete disclosure and the description of how to make and use the present invention, and are not intended to limit the scope of what the inventors regard as their invention nor are they intended to represent that the experiments below are all and only experiments performed. The methodology of preparing few of the preferred embodiments shall become clearer with working examples provided below.

### Example 1—Preparation of Additive

The additive was prepared by dissolving flakes of stearic acid in ethanolamine with continuous stirring to form a solution. After stirring, the prepared solution was subjected to microwave heating for 2-5 minutes at a temperature in the range of 100-180° C. to form an additive in liquid form. Quenching the additive at room temperature to form an additive in solid form. The aforesaid process is portrayed in Scheme 1.

The additive of present invention were prepared by microwave heating. Microwave radiation is electromagnetic radiation and are widely used for synthesis purposes as a source of heating. The technique offers simple, clean, fast, efficient, and economic for the synthesis of a large number of organic molecules. The other advantages of the microwave-assisted method include green synthesis, no waste product, rapid reaction rate and ability to form nanostructures. Moreover, it can be scalable for mass production also.

The additive of present invention was further tested for following tests as described below.

### Example 2—Testing of additive

### 2.1—Analytical Tests

### FTIR Analysis of the Sdditive:

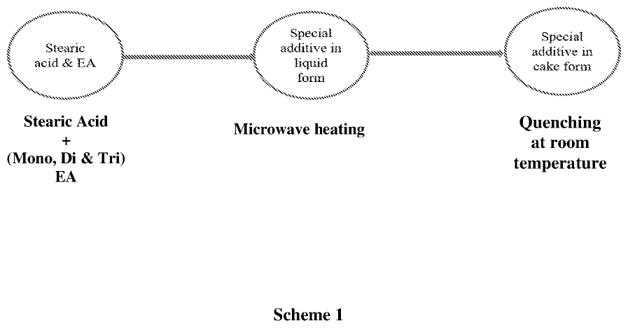

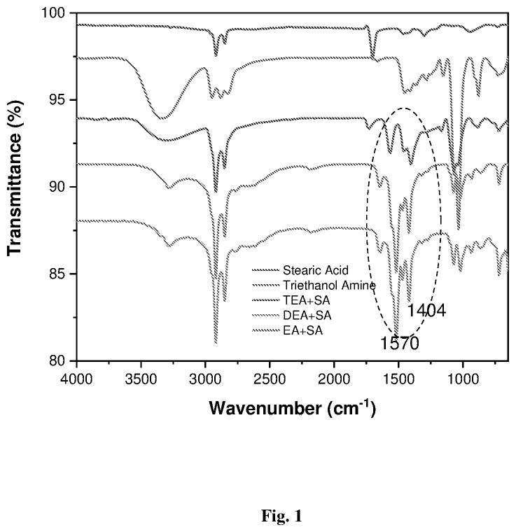

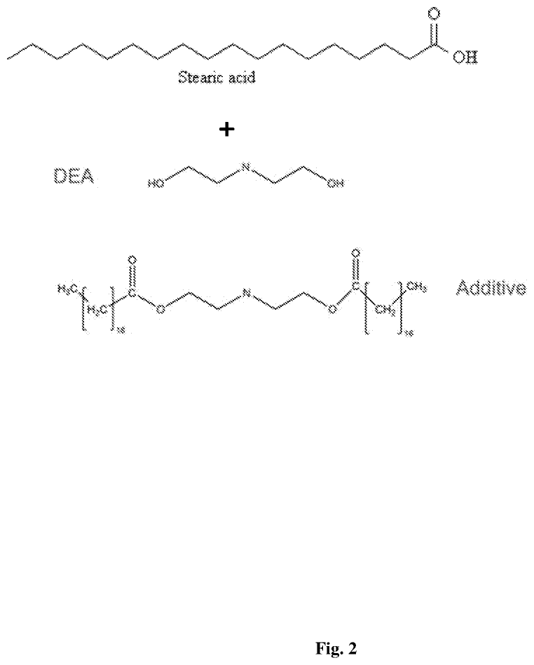

The Fourier-transform infrared (FTIR) studies reveal the peaks on various positions (FIG. 1). The spectrum of additive exhibits an additional peak at 1570 cm⁻¹ & 1404 cm⁻¹ which has been attributed to the ester reaction and N—H bend. All the characteristic peaks demonstrate that the packaging film was successfully fabricated. The spectrum is also provided with respect to the components stearic acid and ethanolamine.

### 2.2—Rubber Compositions

Different rubber compositions were prepared by including the additive and the other components. The results are summarized in Table 1 below.

TABLE 1
Rubber compositions:

| Mixing Stages | Ingredients | Control-A | Control-B | Control-C | DEA1 | DEA2 |
|---|---|---|---|---|---|---|
| Stage 1 | NR | 50.00 | 50.00 | 50.00 | 50.00 | 50.00 |
| | SBR | 50.00 | 50.00 | 50.00 | 50.00 | 50.00 |
| | Silica | 50.00 | 50.00 | 50.00 | 50.00 | 50.00 |
| | Silane | 5.00 | 5.00 | 5.00 | 5.00 | 5.00 |
| Stage 2 | Zinc oxide | | 3.00 | 0.75 | 0.75 | 0.75 |
| | Stearic Acid | | 2.00 | 0.30 | | |
| | Additive | | | | 1.00 | 2.00 |
| | TMQ | 1.00 | 1.00 | 1.00 | 1.00 | 1.00 |
| | 6PPD | 1.00 | 1.00 | 1.00 | 1.00 | 1.00 |
| Stage 3 | Sulphur | 1.40 | 1.40 | 1.40 | 1.40 | 1.40 |
| | TBBS | 1.40 | 1.40 | 1.40 | 1.40 | 1.40 |
| | DPG | 0.25 | 0.25 | 0.25 | | |
| | PVI | 0.20 | 0.20 | 0.20 | 0.20 | 0.20 |

### 2.3—Properties of Rubber Compositions:

The resulted compound compositions exhibit excellent silica dispersions and good physical properties even at lower level of activator ZnO. Compound properties are presented in Table 2. This development offers amended silica dispersion, superior physico-mechanical properties, excellent abrasion resistance, and substantial improvement in cut & chip properties compared to the conventional composite.

TABLE 2
Properties of Rubber compositions:

| | UNITS | Control-A | Control-B | Control-C | DEA1 | DEA2 |
|---|---|---|---|---|---|---|
| tₛ 2 | Mins | 12.45 | 20.53 | 17.45 | 21.55 | 20.43 |
| t′10 | Mins | 5.50 | 16.78 | 14.39 | 9.33 | 14.90 |
| t′25 | Mins | 13.53 | 23.14 | 18.64 | 22.69 | 21.30 |
| t′90 | Mins | 33.72 | 40.76 | 29.33 | 36.08 | 33.98 |
| M_{H} − M_{L} | dNm | 10.16 | 12.71 | 11.11 | 9.99 | 9.85 |
| M_{L} | dNm | 2.68 | 2.24 | 2.76 | 2.80 | 2.71 |
| M_{H} | dNm | 12.85 | 14.94 | 13.87 | 12.79 | 12.56 |
| Hardness | Shore A | 59 | 63 | 62 | 61 | 61 |
| Mooney | MU | 101 | 72 | 101 | 94 | 99 |
| Tensile Strength | kg/cm² | 204 | 243 | 217 | 230 | 241 |
| Elongation at | % | 432 | 442 | 445 | 493 | 507 |
| Break | | | | | | |
| M50 | kg/cm² | 11 | 14 | 11 | 11 | 11 |
| M100 | kg/cm² | 19 | 25 | 19 | 17 | 18 |
| M200 | kg/cm² | 54 | 70 | 53 | 45 | 47 |
| M300 | kg/cm² | 113 | 136 | 114 | 98 | 99 |
| Tear Strength | kg/cm | 38 | 48 | 44 | 43 | 45 |
| Abrasion | mm3 | 78 | 90 | 85 | 72 | 74 |
| Cut &chip | mm3 | 0.87 | 1.31 | 0.95 | 0.93 | 0.98 |
| Toughness | — | 88128 | 107406 | 96565 | 113390 | 122187 |
| Factor | | | | | | |
| Cut &Chip | — | 780 | 790 | 847 | 1157 | 1234 |
| Factor | | | | | | |
| Tan Delta 60° C. | — | 0.160 | 0.130 | 0.146 | 0.153 | 0.154 |
| Tan Delta100° C. | — | 0.137 | 0.103 | 0.122 | 0.130 | 0.129 |
| Payne effect | kPa | 177 | 244 | 384 | 85 | 79 |
| Difference | | | | | | |
| (0.07% to124%) | | | | | | |

Silica has a polar structure and is not compatible with the hydrophobic rubber by nature. Adding additive in the silica loaded compounds significantly improves the dispersion in rubber compounds. Improved dispersion was confirmed Payne effect, Physico-mechanical and abrasion resistance test. In addition to that, the silanol groups on the surface of silica are acidic in nature, and therefore can react with an alkali, such as zinc oxide. This will lead to reduced availability of the silanol groups for reaction with the coupling agent. Therefore, with the use of additive, the applicant have been able to produce rubber compound formulation with significantly reduced dosages of ZnO thereby making the negative influence of ZnO in the silanization reactions as low as possible.

### Advantages of Present Invention

The advantages of the additive are as follows:
Improving the dispersion of silica in rubber compositions.
Reducing amount of zinc oxide in rubber compositions. Rubber compositions including the additive have reduced zinc oxide content up to 75%.
Rubber compositions including the additive have excellent abrasion resistance.
Rubber compositions including the additive have superior cut & chip properties

1. An additive comprising:
stearic acid of formula 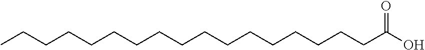 [SMILES: CCCCCCCCCCCCCCCCCC(=O)O]
and ethanolamine, selected from the group consisting of monoethanolamine, diethanolamine and triethanolamine.

2. The additive as claimed in claim 1, wherein the ethanolamine is diethanolamine.

3. A process for the preparation of an additive as claimed in claim 1, wherein said process comprising the steps of:
a) reacting stearic acid with ethanolamine at a temperature in the range of 100-180°° C. for a time period in the range of 2-5 minute to form an additive;
b) quenching of additive of step (a) at room temperature to form an additive in solid form.

4. The process as claimed in claim 3, wherein the temperature in step (a) is 150° C.

5. The process as claimed in claim 3, wherein the time period in step (a) is 3 minute.

6. The process as claimed in claim 3, wherein the amount of stearic acid is in the range of 10% to 100% and ethanolamine is in the range of 10% to 100%.

7. Rubber composition, comprising:
a) Additive as claimed in claim 1, in the range of 0.1-10 parts by weight;
b) polymers in the range of 30-100 part by weight;
c) Silica in the range of 30-90 parts by weight;
d) Silane in the range of 1-10 part by weight.

8. The rubber composition as claimed in claim 7, wherein the polymers is selected from natural rubber, synthetic rubber and other rubbers or a combination of those rubbers.

9. The rubber composition as claimed in claim 7, wherein said composition further comprises:
a) Zinc oxide in the range of 0-5 part by weight;
b) Stearic acid in the range of 0-5 part by weight;
c) TMQ in the range of 0.5-3 part by weight;
d) 6PPD in the range of 0.5-3 part by weight;
e) Sulphur in the range of 0.2-3 part by weight;
f) TBBS in the range of 0.2-3 part by weight;
g) DPG in the range of 0.2-3 part by weight; and
h) PVI in the range of 0.2-3 part by weight.

10. The additive as claimed in claim 1, as and when used for reducing the amount of zinc oxide in rubber compositions to 0-5 part by weight.
